(19) United States
(12) Patent Application Publication
(10) Pub. No.: US 2008/0074753 A1
(43) Pub. Date: Mar. 27, 2008

(54) Compound, Polymer and Optical Component

(75) Inventor: Hiroshi Abe, Otake-shi (JP)

Correspondence Address: FITCH, EVEN, TABIN & FLANNERY, P. O. BOX 18415, WASHINGTON, DC, 20036, US

(73) Assignee: MITSUBISHI RAYON CO., LTD., Minato-ku, Tokyo (JP)

(21) Appl. No.: 11/794,989

(22) PCT Filed: Jan. 12, 2006
(86) PCT No.: PCT/JP06/00262

§ 371 Date: Nov. 20, 2007

(30) Foreign Application Priority Data
Jan. 12, 2005 (JP) 2005-005519
Feb. 25, 2005 (JP) 2005-051795
Apr. 26, 2005 (JP) 2005-127831

(51) Int. Cl. G02B 3/06; C07D 333/64; C08F 28/06

(52) U.S. Cl. 359/642; 526/256; 549/53

(57) ABSTRACT

The present invention relates to a polymer containing a structural unit based on a compound (A-a) represented by the following formula (1-a): 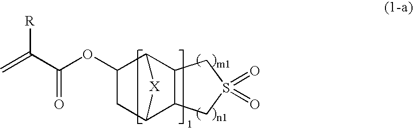
(wherein R represents a hydrogen atom or CH₃, X represents —CH₂—, —O—, or —SO₂—, l represents an integer of 1 or 2, and m1 and n1 each represents an integer of 0 or more, provided that the sum of m1 and n1 is 2), or formula (3): 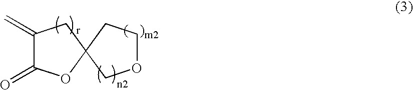
(wherein r represents 1 or 2, m2 represents an integer of 0 to 2, and n2 represents an integer of 1 to 3 provided that the sum of m2 and n2 is 2 or 3, a hydrogen atom in —CH₂— being optionally substituted by another functional group). The polymer may contain a structural unit based on a vinyl monomer (B).

### TECHNICAL FIELD

The present invention relates to a compound having a particular skeleton in a molecule thereof, a polymer obtained by polymerizing the compound, an optical component and a plastic rod lens including the polymer, and a rod lens array in which the plastic rod lenses are arranged.

The present application claims the priority of Japanese Patent Application No. 2005-005519 filed on Jan. 12, 2005, Japanese Patent Application No. 2005-051795 filed on Feb. 25, 2005, and Japanese Patent Application No. 2005-127831 filed on Apr. 26, 2005, the contents of which are incorporated herein by reference.

### BACKGROUND ART

Methacrylic resins such as polymethylmethacrylate are used as lens materials for a camera, video camera, or optical pickup device, optical waveguide materials for an optical fiber, optical connector, rod lens, or the like, due to their high transparency, low birefringence, and balanced characteristics in terms of refractive index, Abbe's number, mechanical property, moldability, weather-resistance, and the like. In particular, a plastic rod lens is used alone, or used in the form of a rod lens array component in which plural rod lenses are integrally arranged in a row as an optical component for an image sensor used in a copying machine, facsimile, scanner, hand scanner, or the like, or as a writing device in an apparatus such as a light-emitting diode (LED) printer utilizing a LED as a light source, a liquid crystal printer utilizing a liquid crystal element, an EL printer utilizing an EL element, or the like.

The polymethylmethacrylate has a refractive index of 1.492 and an Abbe's number of 56. When the polymethylmethacrylate is used as a lens material, the refractive index thereof is not sufficiently high, the curvature of a resultant lens is required to be enlarged, and therefore the lens is excessively thickened. Accordingly, a resin with a high refractive index has been demanded for utilizing as a lens material.

For example, Patent Document 1 discloses a resin obtained by polymerizing thioglycidyl sulfide, the resin having a high refractive index of 1.71 and being useful as a lens material. However, this resin has a low Abbe's number of 36, and therefore may cause a problem such as color blurring due to wavelength dispersion when used as a lens material.

Patent Document 2 discloses a resin obtained by copolymerizing α-methylene-γ-butyrolactone-based compounds. Specifically, a resin with a refractive index higher than that of polymethylmethacrylate is obtained by copolymerizing methyl methacrylate with 3-methylene-dihydrofuran-2-one or 4-methyl-3-methylenedihydrofuran-2-one.

However, although the refractive index of this resin is equal to or greater than that of polymethylmethacrylate, the wavelength dispersion characteristic of the refractive index, that is, Abbe's number, is equal to or slightly lower than that of polymethylmethacrylate.

A plastic rod lens using such a conventional resin exhibits a large chromatic aberration, and therefore cannot realize sufficiently enhanced resolution.

As described above, there are as yet no conventional resins that sufficiently realizes optical characteristics, particularly both high refractive index and high Abbe's number.

Patent Document 1: Japanese Unexamined Patent Application, First Publication No. H9-110979.

Patent Document 2: Japanese Unexamined Patent Application, First Publication No. H8-231648.

### DISCLOSURE OF INVENTION

### Problem to be Solved by the Invention

An object of the present invention is to provide a polymer with a high refractive index and a high Abbe's number, a compound used as a raw material of the polymer, an optical component containing the polymer, a plastic rod lens with an excellent resolution containing the polymer, and a rod lens array in which the rod lenses are arranged.

### Means for Solving the Problem

As a result of earnest investigation for achieving the above-mentioned object, the inventors of the present invention have found that it is possible to realize both high refractive index and high Abbe's number of a polymer containing a structural unit based on a (meth)acrylic acid ester having a heterocyclic skeleton including a sulfonyl group or an exo-methylene lactone having a heterocyclic skeleton including an oxygen atom, and thus the present invention has been completed.

That is, a first aspect of the present invention relates to a compound represented by the following formula (1-a): 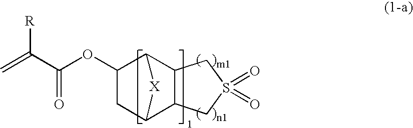
(in which R represents a hydrogen atom or CH₃, X represents —CH₂—, —O—, or —SO₂—, l represents 1 or 2, and m1 and n1 each represents an integer of 0 or more, provided that the sum of m1 and n1 is 2).

A second aspect of the present invention relates to a polymer having a structural unit based on a compound (A-a) represented by the above-mentioned formula (1-a) or the following formula (3): 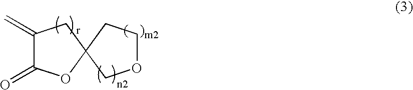
(in which r represents 1 or 2, m2 represents an integer of 0 to 2, n2 represents an integer of 1 to 3, and the sum of m2 and n2 is 2 or 3, and a hydrogen atom of —CH₂— is optionally substituted by another functional group).

A third aspect of the present invention relates to a polymer having the structural unit based on the above-mentioned compound (A-a) and a structural unit based on a vinyl monomer (B).

A fourth aspect of the present invention relates to an optical component such as a plastic rod lens, containing a polymer having a structural unit based on a compound (A) represented by the following formula (1): 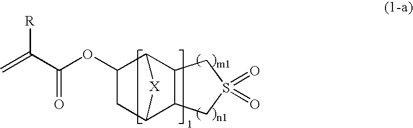
(in which R represents a hydrogen atom or CH₃, X represents —CH₂—, —O—, or —SO₂—, l represents an integer of 0 to 2, and m1 and n1 each represents an integer of 0 or more, provided that the sum of m1 and n1 is 2), or the following formula (3): 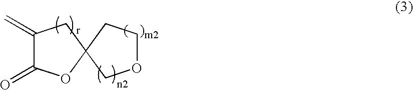
(in which r represents 1 or 2, m2 represents an integer of 0 to 2, n2 represents an integer of 1 to 3, the sum of m2 and n2 is 2 or 3, and a hydrogen atom of —CH₂— is optionally substituted by another functional group).

A fifth aspect of the present invention relates to an optical component and a plastic rod lens, containing a polymer having a refractive index within the range of 1.500 to 1.600 and an Abbe's number within the range of 56 to 70.

A sixth aspect of the present invention relates to a rod lens array including: two base plates; and a plurality of the above-mentioned plastic rod lenses, fixedly arranged in such a way that the plastic rod lenses have a center axis directed almost in parallel with each other between the two base plates.

### EFFECT OF THE INVENTION

Since the polymer according to the present invention has a refractive index and Abbe's number higher that those of polymethylmethacrylate, and the balance between the refractive index and the Abbe's number is favorable, the polymer is excellent as a raw resin used for an optical component such as a plastic lens, plastic optical fiber, plastic rod lens, optical waveguide, or the like, optical resin used for a resin for a disc, a resin for a light-emitting diode, a resin for a transparent electrode, a resin for a liquid crystal display, or the like. The compound according to the present invention can be used as a raw material of such a polymer.

Also, the rod lens array in which the rod lenses containing such a polymer are fixedly arranged has a high resolution, and therefore, the rod lens array is particularly suitable for a copying machine, facsimile, printer, or scanner.

### BRIEF DESCRIPTION OF DRAWINGS

FIG. 1 is a perspective view illustrating a step of winding a rod lens according to the present invention around a cylindrical roll.

### EXPLANATION OF NUMERALS

1 Cylindrical roll
2 Base plate
3 Adhesive
4 Rod lens
5 Dancer guide
6 Self-mobile guide

### BEST MODE FOR CARRYING OUT THE INVENTION

In the following, the present invention will be circumstantially explained. In the present specification, the term “(meth)acrylic acid” is a general term meaning acrylic acids and methacrylic acids.

The above-mentioned compound (A-a) is a (meth)acrylic acid ester having a heterocyclic skeleton including a sulfonyl group or an exo-methylene lactone having a heterocyclic skeleton including an oxygen atom.

First, the (meth)acrylic acid ester having a heterocyclic skeleton including a sulfonyl group (that is, the compound represented by the above-mentioned formula (1-a)) will be explained.

If polymers and optical resins have the heterocyclic skeleton including a sulfonyl group, they exhibit an enhanced refractive index and Abbe's number and improved wavelength dispersion. Moreover, their (meth)acrylic acid ester portion is excellent in polymerizability and also in copolymerizability with a monomer such as methyl methacrylate (MMA).

Among them, the compounds represented by the above-mentioned formula (1-a), in which 1 represents 1 or 2, are preferable in that the balance between moisture (water) absorption properties and optical characteristic thereof is favorable. Among them, the compounds in which X represents —CH₂— are preferable. Also, it is required in the formula (1-a) that the sum of m1 and n1 be 2 so as to realize high refractive index and Abbe's number, easy synthesis, and stabilized compounds.

Examples of the compound (A-a) represented by the above-mentioned formula (1-a) include 1,1-dioxidetetrahydrothiene-3-yl(meth)acrylate, 1,1-dioxidetetrahydrothiene-2-yl(meth)acrylate, 4,4-dioxide-4-thiatricyclo[5.2.1.0^{2,6}]dec-8-yl(meth)acrylate, 3,3-dioxide-3-thiatricyclo[5.2.1.0^{2,6}]dec-8-yl(meth)acrylate, 3,3-dioxide-3-thiatricyclo[5.2.1.0^{2,6}]dec-9-yl(meth)acrylate, 6,6-dioxide-6-thiapentacyclo[9.2.1.0^{2,10}.1.0^{4,8}]pentadec-12-yl(meth)acrylate, 5,5-dioxide-5-thiapentacyclo[9.2.1.0^{2,10}.1.0^{4,8}]pentadec-12-yl(meth)acrylate, 5,5-dioxide-5-thiapentacyclo[9.2.1.0^{2,10}.1.0^{4,8}]pentadec-13-yl(meth)acrylate, and the like. These may be used alone or in combination thereof.

1,1-Dioxidetetrahydrothiene-3-yl(meth)acrylate and 1,1-dioxidetetrahydrothiene-2-yl(meth)acrylate, which are compounds (A-b) represented by the following formula (1-b), are preferable in terms of their excellent compatibility with polymethylmethacrylate. 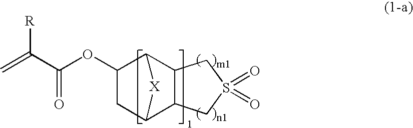
(In the formula, R represents a hydrogen atom or CH₃, X represents —CH₂—, —O—, or —SO₂—, m1 and n1 each represents an integer of 0 or more, and the sum of m1 and n1 is 2.)

4,4-Dioxide-4-thiatricyclo[5.2.1.0^{2,6}]dec-8-yl(meth)acrylate, 3,3-dioxide-3-thiatricyclo[5.2.1.0^{2,6}]dec-8-yl(meth)acrylate, 3,3-dioxide-3-thiatricyclo[5.2.1.0^{2,6}]dec-9-yl(meth)acrylate, 6,6-dioxide-6-thiapentacyclo[9.2.1.0^{2,10}.1.0^{4,8}]pentadec-12-yl(meth)acrylate, 5,5-dioxide-5-thiapentacyclo[9.2.1.0^{2,10}.1.0^{4,8}]pentadec-12-yl(meth)acrylate, 5,5-dioxide-5-thiapentacyclo[9.2.1.0^{2,10}.1.0^{4,8}]pentadec-13-yl(meth)acrylate, and the like, are preferable, in that the refractive index of the polymer is enhanced and the water-absorbability thereof is decreased.

The variation of transparency, refractive index, and Abbe's number, which are caused by water-absorption, can be suppressed by decreasing the water-absorbability of the polymer.

Methods for producing the compound (A-a) represented by the formula (1-a) are not particularly limited. For example, Izobreteniya, 1998, (34), 333, discloses that 1,1-dioxidetetrahydrothiene-3-ylmethacrylate is prepared by reacting 3-hydroxysulfolane with methacrylic acid.

Also, 3-thiatricyclo[5.2.1.0^{2,6}]dec-8-ene 3,3-dioxide is synthesized by subjecting 2-sulfolene and cyclopentadiene to a Diels-Alder reaction, and then reacted with a methacrylic acid to synthesize 3,3-dioxide-3-thiatricyclo[5.2.1.0^{2,6}]dec-8-ylmethacrylate or 3,3-dioxide-3-thiatricyclo[5.2.1.0^{2,6}]dec-9-ylmethacrylate. Instead of the methacrylic acid, an acrylic acid may be used to synthesize 3,3-dioxide-3-thiatricyclo[5.2.1.0^{2,6}]dec-8-ylacrylate or 3,3-dioxide-3-thiatricyclo[5.2.1.0^{2,6}]dec-9-ylacrylate. Other kinds of the compound (A-a) can be synthesized by a similar method.

Next, the exo-methylene lactone having a heterocyclic skeleton including an oxygen atom (that is, the compound represented by the above-mentioned formula (3)) will be explained.

If polymers and optical resins have the heterocyclic skeleton including an oxygen atom, both the refractive index and Abbe's number are enhanced, and the chromatic aberration is reduced. The compounds (A-a) and (A), which are represented by the formula (3), have a cyclic skeleton, and exhibit a refractive index and Tg higher than those having a chain skeleton. Also, an exo-methylene is excellent in polymerizability and also in copolymerizability with a monomer such as MMA.

In the formula (3), r represents 1 or 2, m2 represents an integer of 0 to 2, n2 represents an integer of 1 to 3, and the sum of m2 and n2 is 2 or 3, in view of ease of synthesis and stability of the compounds. In that the Abbe's number and refractive index are enhanced, r is preferably 1 in the formula (3). Also, a hydrogen atom in —CH₂— forming a ring may be substituted by a functional group such as a hydroxyl group, alkoxy group, cyano group, or the like.

The compound represented by the formula (3) forms a structural unit represented by the following formula (3p) by polymerization. 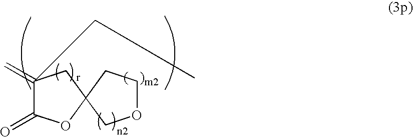
(In the formula, r represents 1 or 2, m2 represents an integer of 0 to 2, n2 represents an integer of 1 to 3, the sum of m2 and n2 represents 2 or 3, and a hydrogen atom in —CH₂— is optionally substituted by another functional group.)

The compounds (A-a) or (A), represented by the formula (3), can be identified based on ¹H-NMR spectrum data disclosed in Phosphorus and Sulfur, 1984, 19, 137, N. Satyamurthy et al., (Non-patent Document 1).

Examples of the compounds (A-a) and (A), represented by the formula (3), include 3-methylene-1,8-dioxaspiro[4,5]decan-2-one, 3-methylene-1,7-dioxaspiro[4,5]decan-2-one, 3-methylene-1,7-dioxaspiro[4,4]decan-2-one, and the like. Among these, 3-methylene-1,8-dioxaspiro[4,5]decan-2-one is preferable due to its ease of synthesis.

The compounds (A-a) and (A) represented by the formula (3) may be synthesized by any methods, For example, 1,6-dioxaspiro[2,5]octane and diethyl malonate are reacted to synthesize 1,8-dioxaspiro[4,5]decan-2-one as an intermediate, followed by introducing a methylene group to obtain 3-methylene-1,8-dioxaspiro[4,5]decan-2-one, as disclosed in Non-patent Document 1. Also, 3-methylene-1,7-dioxaspiro[4,5]decan-2-one, 3-methylene-1,7-dioxaspiro[4,4]decan-2-one, and the like can be synthesized in a similar manner to the above.

Polymers obtained by polymerizing at least one of the compounds (A-a) and (A) are resins with a refractive index within the range of 1.500 to 1.600 and an Abbe's number within the range of 56 to 70, and thus both of the refractive index and Abbe's number thereof are higher than those of polymethylmethacrylate. Plastic rod lenses including such a polymer exhibit low chromatic aberration and high resolution.

Examples of monomers producing the polymers with a refractive index within the range of 1.500 to 1.600 and an Abbe's number within the range of 56 to 70 by polymerization include α-trifluoromethylacrylic acid esters (D) represented by the following formula (4): 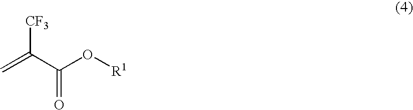
(in which R¹ represents a five- to ten-membered alicyclic hydrocarbon group).

In the formula (4), an ester portion R¹ is a five- to ten-membered alicyclic hydrocarbon group. Examples thereof include an organic group having an adamantyl skeleton. Specific examples thereof include a 1-adamantyl group, 2-adamantyl group, 2-methyl-1-adamantyl group, 2-methyl-2-adamantyl group, 1-hydroxy-2-adamantyl 3-hydroxy-1-adamantyl group, and the like.

The monomers (D) may be used alone or in combination of at least two kinds thereof.

The monomers (D) may be synthesized by any methods. For example, α-trifluoromethylacrylic acid chloride and 1,3-adamantanediol are reacted at −10° C., followed by performing purification using a silica gel column chromatography to obtain a 1-adamantyl-3-hydroxy-α-trifluoromethyl acrylate, as disclosed in Japanese Laid-Open Patent Application No. 2003-137841.

According to the present invention, the polymers may be composed of a structural unit based on the compound (A-a) or (A) only, however, the polymers may include another structural unit based on a vinyl monomer (B) or the like so as to adjust the refractive index.

Examples of the vinyl monomer (B) available according to the present invention include (meth)acrylic acid esters (however, excepting the compounds represented by the formula (A-a) or (A)), styrene, (meth)acrylonitrile, vinyl acetate, ethylene, butadiene, methylvinylketone, (meth)acrylamide, vinylidene chloride, tetrafluoroethylene, maleic anhydride, and the like. Among these, (meth)acrylic acid esters, particularly methacrylic acid esters, are preferably used in terms of their transparency, thermal resistance, and the like. These may be used in combination with at least two kinds thereof.

Among the vinyl monomers (B) available according to the present invention, compounds represented by the following formula (5), for example, may be used as the (meth)acrylic acid esters.

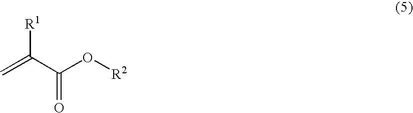

In the formula (5), R¹ represents a hydrogen atom or a methyl group. R² represents a C1 to C20 alkyl group, C3 to C20 cycloalkyl group, or five- to ten-membered group excepting the above-mentioned cycloalkyl groups. The five- to ten-membered group contains a heterocyclic ring including at least one oxygen atom. Also, the five- to ten-membered group may be composed of not only a monocyclic ring, but also a polycyclic ring such as a skeleton derived from a bicyclo[2.2.1]heptane, tetracyclo[4.4.0.1^{2,5}.1^{7,10}]dodecan, or the like.

Specific examples thereof include alkyl (meth)acrylates such as methyl (meth)acrylate, butyl (meth)acrylate, isobutyl (meth)acrylate, t-butyl (meth)acrylate, cyclohexyl (meth)acrylate, ethylhexyl (meth)acrylate, n-octyl (meth)acrylate, isooctyl (meth)acrylate, nonyl (meth)acrylate, tridecyl (meth)acrylate, lauryl (meth)acrylate, isobornyl (meth)acrylate, tetrahydrofurfuryl (meth)acrylate, 2,2,3,3-tetrafluoropropyl (meth)acrylate, 2,2,3,3,4,4,5,5-octafluoropentyl (meth)acrylate, 2,2,2-trifluoroethyl (meth)acrylate, and the like; phenyl (meth)acrylate, benzyl (meth)acrylate, tricyclo[5.2.1.0^{2,6}]decanyl (meth)acrylate, and the like. These may be used in combination of at least two kinds thereof.

It is preferable that the copolymerization ratio of the compound (A-a) or (A) to the vinyl monomer (B) in a copolymer, which is determined by the following formula:

{the content of the compound (A-a) or (A)}×100/[{the content of the compound (A-a) or (A)}+the content of the vinyl monomer (B)] (% by mass), be within the range of 1 to 100% by mass, more preferably 10 to 100% by mass, and even more preferably 20 to 100% by mass. A molded product with an improved refractive index and Abbe's number can be provided by increasing the copolymerization ratio.

The polymer according to the present invention may optionally contain a dye or the like.

Methods for polymerization of the polymer according to the present invention are not particularly limited, and well-known methods such as radical polymerization, ion polymerization, coordination polymerization, or the like, may be adopted. Among these, radical polymerization is preferably adopted. The polymerization form may be any of bulk polymerization, solution polymerization, suspension polymerization, emulsion polymerization, and the like, and may be performed by any of a batch operation, semicontinuous operation, and continuous operation. Also, either thermal polymerization or photo polymerization may be adopted.

The temperature for performing polymerization reaction is suitably determined in consideration of the polymerization process, polymerization form, kinds of initiator, and the like. Generally, it is preferable that the temperature be within the range of 20 to 200° C., and particularly preferable within the range of 50 to 140° C.

A reactor used for performing the polymerization reaction is not particularly limited.

As a polymerization solvent used for performing the solution polymerization reaction, ones which do not inhibit polymerization are preferable, and examples thereof include: ketone solvents such as acetone, methylisobutylketone, and the like; aromatic solvents such as toluene, xylene, benzene, and the like; cyclic hydrocarbon solvents such as cyclopentane, cyclohexane, and the like; alcohol solvents such as isopropyl alcohol, ethyleneglycol monomethyl ether, and the like; ester solvents such as methyl acetate, ethyl acetate, and the like. These may be used in combination of at least two kinds thereof.

The solvent is preferably determined in consideration of the solubility of the monomer used and the solubility of the polymer produced. Also, a chain transfer agent such as mercaptan may be used together.

A radical polymerization initiator used for performing radical polymerization is not particularly limited, and examples thereof include azo compounds such as 2,2′-azobisisobutylonitrile, dimethyl-2,2′-azobisisobutylate, and the like; peroxides such as hydrogen peroxide, cumene hydroperoxide, benzoyl peroxide, t-butyl peroxide, and the like; persulfates such as ammonium persulfate, potassium persulfate, sodium persulfate, and the like; redox initiators such as mixtures of the above-mentioned initiator and a reductant such as sodium sulfite, sodium thiosulfate, or the like; initiators in which a small amount of a metal such as iron, a ferrous salt, silver sulfate, copper sulfate, or the like coexists with the above mixtures, and the like. These may be used in combination of at least two kinds thereof. These radical initiator may be added at once upon start of polymerization or may be added in increments during polymerization.

Examples of an emulsifier available for performing emulsion polymerization include anionic emulsifiers and nonionic emulsifiers. Examples of the anionic emulsifiers include alkylbenzene sulfonates, alkyl sulfates, and derivatives thereof. Examples of the nonionic emulsifiers include polyoxyethylene alkylphenol ethers, polyoxyethylene alkyl ethers, and the like. These may be used in combination of at least two kinds thereof.

A radical polymerization initiator used for performing emulsion polymerization is not particularly limited, provided that it is used for performing general emulsion polymerization. Among them, water-soluble initiators are particularly preferable.

As a method for polymerization, anionic polymerization may be adopted. The polymerization form may be bulk polymerization or solution polymerization. An initiator used for performing anionic polymerization is not particularly limited, and any ones generally used may be used. Examples thereof include organic lithium compounds such as n-butyl lithium, t-butyl lithium, and the like. Also, metallic alkoxides such as sodium methoxide, potassium methoxide, sodium t-buthoxide, potassium ethoxide, sodium t-buthoxide, potassium t-buthoxide, or the like, are preferably used. Also, a nitrogen-containing heterocyclic compound such as pyridine, picoline, piperidine, or the like; or an amine such as triethylamine, tributylamine, triethanolamine, or the like, is preferably used. These may be used in combination of at least two kinds thereof.

As a method for removing an organic solvent or water contained as a medium from a solution or dispersion prepared in such a way, any well-known methods may be adopted. For example, a method in which filtration or distillation by heating under reduced pressure is performed after reprecipitation may be adopted.

The number average molecular weight of the polymer according to the present invention is generally within the range of 1,000 to 1,000,000, and more preferably 10,000 to 500,000. When the molecular weight is 1,000 or more, mechanical properties of a molded product can be sufficiently increased. When the molecular weight is 1,000,000 or less, the solvent solubility can be improved.

In the following, a rod lens and a rod lens array according to the present invention will be explained.

The rod lens according to the present invention is a cylindrical lens with a refractive index distribution in which the refractive index thereof continuously decreases from the center to the outer circumference. In this refractive index distribution, it is preferable that a refractive index distribution in the region where the distance from the center axis to the outer circumference direction is 0.3r to 0.7r (r represents a radius of the rod lens measured in a section intersecting perpendicularly with a center axis of the rod lends) be approximate to a secondary curve distribution defined by the following formula (X):
n(L)=n₀{1−(g²/2)L²} (X)
(in which no represents a refractive index at the center axis of the rod lens (central refractive index), L represents a distance from the center axis of the rod lends (0≦L≦r), g represents a distribution constant of the refractive index of the rod lens, and n(L) represents a refractive index at a position with a distance L from the center axis of the rod lens).

Although the radius r of the rod lens according to the present invention is not particularly limited, it is preferable that the radius r be small from the standpoint of compactification of an optical system, but the radius r be large from the standpoint of ease of manipulation. Accordingly, it is preferable that the radius r of the rod lens be within the range of 0.05 to 1 mm.

Also, it is preferable that the refractive index no of the center axis of the rod lens be within the range of 1.4 to 1.6 in that options of materials composing the rod lens are widened and a favorable refractive index distribution is easily realized. It is preferable that the polymer according to the present invention be used for the center axis of the rod lens according to the present invention due to its high refractive index and Abbe's number.

Moreover, although the distribution constant g of the refractive index of the rod lens is not particularly limited, it is preferable that the distribution constant g be within the range of 0.2 to 3 mm⁻¹, and more preferably 0.5 to 2 mm⁻¹, from the standpoint of compactification of an optical system, securing of operating distance of an optical system, and ease of manipulation.

It is preferable that a light absorbing layer containing a light absorber for absorbing at least a part of light transmitting in the rod lens be provided at an outer circumference portion with a distance of 0.6r or more from the center axis of the rod lens according to the present invention. This aims to suppress deterioration of optical characteristics caused by the existence of irregular portions in which the refractive index distribution deviates greatly from an ideal distribution, the irregular portions being easily formed in the rod lens outward from the center axis thereof in general, by providing the light absorbing layer at the outer circumference portion of the rod lens. It is preferable that the thickness of the light absorbing layer be within the range of 50 to 100 μm. If the thickness of the light absorbing layer is within this range, flare light and crosstalk light can be sufficiently removed, and a sufficient light transmission quantity can be secured.

In the following, a method for producing the above-mentioned rod lens will be explained.

First, N uncured objects with each refractive index n measured after being cured, n1 to nN (N≧3, n1>n2> . . . >nN), are concentrically laminated so that the refractive index gradually decreases from the center to the outer circumference to obtain an uncured laminate (hereinafter, referred to as “filament”). Then, the filament is cured while or after subjecting components between adjacent layers to a mutual diffusion processing so that the refractive index distribution between each layers of this filament continuously changes, to obtain an original rod lens yarn. The term “mutual diffusion processing” means a processing in which a thermal history is imparted to the filament under a nitrogen atmosphere at 10 to 60° C., more preferably 20 to 50° C., for several seconds to several minutes. As needed, the obtained original rod lens yarn may be heated and drawn, followed by performing relaxing processing. The original rod lens yarn produced in such a way is suitably cut into a predetermined size to obtain a rod lens.

As materials composing the uncured objects, compositions containing the above-mentioned compound (A-a) or (A) and/or the above-mentioned vinyl monomer (B) may be used.

It is preferable that the uncured objects be composed of the composition containing the above-mentioned compound (A-a) or (A) and/or the above-mentioned vinyl monomer (B) and a soluble polymer soluble therein, so as to facilitate viscosity control of the uncured objects at the time of forming the filament from the uncured objects and realize continuous refractive index distribution from the central to the outer circumference of the filament.

As the soluble polymer required to be compatible with the polymer according to the present invention, a polymer having a structural unit based on the compound (A-a) or (A) and/or a structural unit based on the vinyl monomer (B) is preferably used. In addition to the above, polymethylmethacrylates, polymethylmethacrylate-based copolymers, poly4-methylpentene-1, ethylene/vinyl acetate copolymers, polyvinylidene-fluoride, vinylidene-fluoride/tetrafluoroethylene copolymers, vinylidene-fluoride/tetrafluoroethylene/hexafluoropropylene copolymers, or the like may be used, for example. In addition to the above, polycarbonates or the like may be used.

In order to cure the filament formed using the uncured objects, heating processing and/or photosetting processing is performed after a thermosetting catalyst or photosetting catalyst is added to the uncured objects. As the thermosetting catalyst, a peroxide-based catalyst, an azo-based catalyst, or the like, may be used.

The photosetting processing may be performed by exposing the uncured objects containing the photosetting catalyst to ultraviolet rays from the circumference thereof, for example. As a light source to be used for the photosetting processing, a carbon arc lamp emitting light with a wavelength of 150 to 600 nm, a high-pressure mercury lamp, a medium-pressure mercury lamp, a low-pressure mercury lamp, an ultrahigh-pressure mercury lamp, a chemical lamp, a xenon lamp, laser light, or the like may be used. In order to increase the polymerization rate, these light sources may be suitably combined for use.

It is preferable that the thermosetting processing be performed by subjecting the uncured objects containing the thermosetting catalyst to heating processing in a curing part such as a heating furnace controlled to maintain a constant temperature, for example.

The original rod lens yarn produced in such a way may be continuously cut into a predetermined length, or may be cut after being wound around a bobbin or the like.

After that, the rod lenses are arranged to produce a rod lens array according to the present invention. The rod lens array according to the present invention is formed by arranging plural rod lenses in at least a row between two base plates so that each rod lens has an optical axis directed almost in parallel with each other. An adhesive is used for fixing the rod lenses to the base plates. The adjacent rod lenses may contact with each other or may be separated with constant intervals. If the rod lens array is formed by the same kind of the rod lenses arranged and laminated in at least two layers, it is preferable that the rod lenses be arranged in a straw bag stacking state so that the space between the rod lenses is minimum.

The base plate composing the rod lens array according to the present invention may be a flat plate or may be equipped with a U-shaped or V-shaped groove for arranging and storing the rod lenses with constant intervals. Although a material used for composing the base plate is not particularly limited, ones that can be easily processed for producing the rod lens array are preferable. As the material used for composing the base plate, various thermoplastic resins and various thermosetting resins are preferable, and phenol resins, ABS resins, polyimide-based resins, liquid crystal polymers, epoxy-based resins, and the like, are particularly preferable.

As a method for arranging the rod lenses, the following methods may be adopted.

<Method 1>

First, plural rod lenses cut into a constant length are arrayed closely to each other or separately with constant pitches on an array tool equipped with a suction mechanism so that each center axis of the rod lenses is directed in parallel to each other to form a rod lens row (step of arraying rod lenses).

Examples of the array tool equipped with a suction mechanism include ones composed of a flat plate having a hole part or a groove part connected to a suction apparatus such as a vacuum pump or the like or composed of a member having a V-shaped or U-shaped groove, or the like, for storing lenses with constant pitches, and ones having a structure which enables to array the rod lenses parallel to each other closely or separately with constant pitches on the flat plate or the member by utilizing a sucking force from the hole part or the groove part connected to the suction apparatus.

Next, plural rod lenses are arrayed in a straw bag stacking state on the rod lens row (the first row) arrayed on the array tool in the same manner so as to form a second rod lens row. At that time, the second rod lens row is sucked and supported from fine spaces between the rod lenses of the first row.

Next, a first base plate having one surface on which an adhesive is applied is prepared. The first base plate is adhered to the second rod lens row on the array tool via the adhesive applied on the first base plate so as to fixedly adhere the second rod lens array on the first base plate (step of fixing the first base plate).

Check plates are provided on both side end portions (side end portions in a center axis direction of the rod lenses) of the first base plate on which the second rod lens row is adhered. Instead of the check plates, rod lenses located at side end portions of the rod lens row on the first base plate may be fixed on the first base plate as stoppers.

Next, an adhesive is applied on the rod lens row adhered on the first base plate, and the first rod lens row on the array tool is fixedly adhered in a straw bag stacking state on the rod lens row adhered on the first base plate.

Next, a second base plate having one surface on which an adhesive is applied is prepared. The second base plate and the second rod lens row adhered on the first base plate are adhered together via the adhesive applied on the second base plate (step of fixing the second base plate).

Then, one side end of the rod lens rows held between the first base plate and the second base plate is contacted to an uncured liquid adhesive containing a light shielding agent such as carbon black, dye, or the like, followed by decompressing the other side end thereof so as to fill inside gaps with the adhesive. Then, the filling adhesive is cured to form an original rod lens array plate.

Although the case in which the rod lenses are arrayed in two rows (two layers) between the two base plates is described in the above, the number of rod lens rows arrayed between the two base plates may be one (one layer) or at least three (at least three layer) in the present invention.

<Method 2>

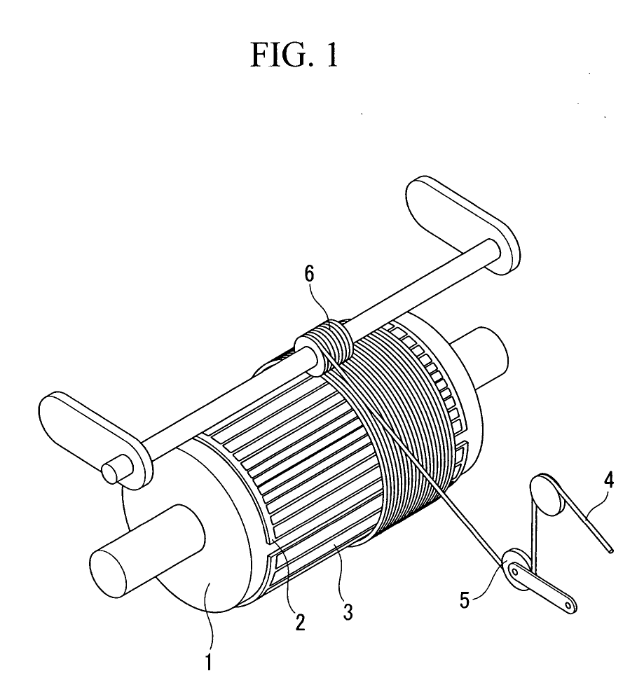

In the following, Method 2 will be explained with reference to FIG. 1. FIG. 1 is a drawing illustrating a step of winding a rod lens around a base plate held by a cylindrical roll.

A base plate 2 (first base plate) on which an adhesive 3 is applied is fixed while being wound around a cylindrical roll 1 having a function of adhering the base plate. A method for fixing the base plate 2 around the cylindrical roll 1 is not particularly limited, provided that the base plate 2 is sufficiently fixed around the cylindrical roll 1 without the base plate 2 falling from the cylindrical roll 1 during winding a rod lens 4 and without deviation occurring between the roll 1 rotated and the base plate 2, and that the base plate 2 is easily removed from the cylindrical roll 1 after being fixed in such a way. For example, the base plate 2 may be fixed by decompressing to suck the base plate 2 from the cylindrical roll 1, applying an adhesive on the surface of the cylindrical roll 1, adhering an adhesive sheet on the surface of the cylindrical roll 1, or the like. The adhesive 3 may be applied on the base plate 2 after the base plate 2 is fixed on the cylindrical roll 1. The rod lens 4 may be fixedly adhered to the base plate 2 by applying the adhesive 3 on the base plate 2 or arraying an adhesive sheet on the base plates, followed by winding the rod lens 4 thereon.

It is preferable that the base plate 2 have a flexibility with a function as a base plate of an optical transmission member so as to fix the base plate 2 by winding it around the cylindrical roll 1. A plastic base plate, such as, for example, BAKELITE (phenol resin), or the like, is preferably used. The shape of the base plate 2 may be rectangular, and the size of the base plate 2 is suitably determined depending on the desired array width or the size of the cylindrical roll 1. If the base plate 2 is rectangular, the length of the base plate 2 in the outer circumference direction (vertical direction with respect to a rotation axis of the roll) of the cylindrical roll 1 is preferably equal to or shorter than that of the cross-sectional circumference of the cylindrical roll 1, and is preferably equal to the length which enables a pair of opposite sides thereof along to a direction of the rotation axis of the roll to join together or approximately join together on the surface of the outer circumference of the cylindrical roll 1 when the base plate 2 is wound around the cylindrical roll 1.

It is preferable that the outer circumference of the cylindrical roll 1 have a large curvature radius, so as to facilitate winding of the base plate 2, increase the number of optical transmission arrays which can be cut off from one original optical transmission array plate (productivity), and suppress warpage of the base plate separated from the cylindrical roll 1.

The rod lens 4 is wound through a guide around the base plate 2 fixed on the cylindrical roll 1 by rotating the cylindrical roll 1 using a torque motor or the like. It is preferable that the tension applied on the rod lens 4 at that time be constant so as to wind the rod lens 4 uniformly. The tension applied on the rod lens 4 may be controlled using a dancer guide 5 for controlling a torque (dancer roll), a tensionmeter, or the like, and thus a well-known tension controller may be suitably adopted. It is preferable that the tension at the time of winding the rod lens 4 be adjusted within the range between 0.29 N and 1.96 N. If the tension is extremely small, wind-collapse easily occurs. If the tension is extremely large, the base plate 2 separated from the cylindrical roll 1 easily warps due to influence of stress remaining in the optical transmission member.

Also, the rod lens 4 is wound so that optical transmission members adjacent in the same array layer form gaps therebetween. The gaps may be filled with an opaque adhesive, and thereby, cross-talk between the optical transmission members can be suppressed. It is preferable that the distance between the optical transmission members be within the range of 2 to 50 μm. If the distance is extremely small, sufficient effects of suppressing cross-talk cannot be realized. On the other hand, if the distance is extremely large, that is, the adjacent optical transmission members are greatly spaced, the optical characteristic (such as quantity of light or the like) as an optical transmission array deteriorates.

The rod lens 4 is wound so that the distance (pitch) between center axes of adjacent optical transmission members is constant. By forming gaps between the adjacent optical transmission members in the same array layer, the distance between the center axes of the optical transmission members is not influenced by the variation of an external diameter of the optical transmission member, and thereby the optical transmission members can be arrayed with constant intervals even if the optical transmission members have some diameter variance. That is, the array pitch variance of the optical transmission members in the optical transmission array can be decreased, and thus the optical transmission array with excellent optical characteristic can be produced.

In FIG. 1, a method for winding the rod lens 4 with constant intervals using a self-mobile guide 6 for winding is illustrated. The self-mobile guide 6 is a cylindrical body in which grooves, each having a width for storing one optical transmission member, are spirally formed, the pitch of the groove being set to be slightly larger than the diameter R of the rod lens 4. By adjusting the size of the pitch, the separation of the wound rod lens 4 can be controlled. The rod lens 4 wound around the cylindrical roll 1 serves as a lead screw with respect to the self-mobile guide 6, and thereby the self-mobile guide 6 moves in an axial direction of the cylindrical roll 1 by one pitch of the groove provided on the self-mobile guide 6, as a result of which the rod lens 4 is moved (traversed) with a constant width in an axial direction of the cylindrical roll 1.

Although the self-mobile guide 6 is used so as to simplify the device in the above, a method for traversing is not particularly limited provided that the guide is traversed with constant intervals in the method. A linear motion, a linear motor, or the like may be used.

The rod lens 4 may be wound to a predetermined array width to obtain a winding body with a layer. Moreover, the rod lens 4 may be further wound on the winding body with a layer to obtain a winding body with at least two layers. In the latter case, the second layer of the winding body is formed by applying an adhesive on the first layer of the winding body, followed by winding the rod lens 4 further supplied so as to be placed on the gaps between optical transmission members of the first layer in a straw bag stacking state. By repeatedly performing the same procedure thereafter, a winding body of the optical transmission member arrayed in predetermined layers is produced. Such a winding body with plural layers can be produced by winding the rod lens 4 around the roll with intervals less than twice the diameter thereof. The second or the subsequent layer of the winding body may be fixedly adhered by applying the adhesive 3 on the winding body or placing an adhesive sheet on the winding body, followed by winding the rod lens 4 thereon.

After the winding body of the optical transmission member arrayed in predetermined layers is formed, the winding body of the rod lens 4 wound around the cylindrical roll 1 is cut in a direction of the rotation axis of the cylindrical roll 1. This cutting procedure may be performed along the end portions, that is, discontinuous portions, of the base plate 2 wound around the cylindrical roll 1 in an outer circumference direction of the roll. For example, if the base plate 2 wound around the cylindrical roll 1 is rectangular, it is preferable that the cutting procedure be performed along the portion in which a pair of the opposite sides along a direction of the rotation axis of the roll is joined together or approximately joined together on the outer circumference of the cylindrical roll 1. After performing the cutting procedure, the base plate 2 is separated from the cylindrical roll 1, and thus an array of the rod lenses 4 provided on the base plate 2 is obtained.

On the array formed on the base plate 2, the adhesive 3 is applied, and another base plate (second base plate) (which is not shown in the drawing) is placed thereon, followed by fixedly adhering the array between the two base plates, and then curing the adhesive 3 to obtain an original rod lens array plate. The second base plates may also be fixedly adhered by applying an adhesive on the array or placing an adhesive sheet on the array, followed by pressing the second base plates thereon.

After the original rod lens array plate prepared in accordance with the above-mentioned method 1 or 2, or the like, is cut to a predetermined length, both end portions of the rod lens 4 are mirror-finished using a diamond blade or the like to produce a rod lens array. If the length of the rod lens 4 used is approximately the same as that of the rod lens array, the original rod lens array plate need not be cut.

The adhesive to be used is not particularly limited, provided that the adhesive has a sufficient adhesive power for adhering the rod lens layers on the base plates or rod lens layers together. As the adhesive, an adhesive that can be applied in a thin film, a spray-type adhesive, a hot-melt type adhesive, or the like may be used.

### EXAMPLES

In the following, the present invention will be further circumstantially explained by showing examples. However, the present invention is not limited by these examples.

In the following examples, measurements of physical properties of polymers and the like were performed in accordance with the following methods. As samples for measuring the refractive index and Abbe's number, a chloroform or dimethyl sulfoxide solution containing 10% by mass of each polymer was prepared, cast in a laboratory dish, dried at room temperature over night, and then further dried in a vacuum for 24 hours to obtain a cast film, the cast film being cut into a form suitable for measurement, as needed, to be used.

<Measurement of Refractive Index and Abbe's Number>

The refractive index was measured at a temperature of 25° C. and a wavelength of 589 nm using an Abbe's refractometer manufactured by ATAGO CO., LTD. The Abbe's number was determined by measuring the refractive index at a temperature of 25° C. and wavelengths of 486, 589, and 656 nm.

<Relative Proportions of Polymer>

The relative proportions of the polymer were determined by measuring ¹H-NMR (270 MHz).

<Water Absorption Percentage>

The percentage of water absorption was determined by measuring the mass of each test piece before and after immersing the test piece in water for 24 hours.

### Example 1

### Synthesis of 2,3-dihydrothiophene 1,1-dioxide

A three-necked flask equipped with a reflux cooling tube, a stirrer bar, and a thermometer was charged with 100 g (0.85 mol) of 2,5-dihydrothiophene 1,1-dioxide, followed by heating to 66° C., and then stirring for 30 minutes. Then, 28.5 g (0.25 mol) of potassium t-buthoxide was added and reacted for 6 hours. After the reaction was ended, the reactant was cooled to room temperature, and then neutralized by adding 35% hydrochloric acid. The reactant was evaporated using an evaporator to remove t-butanol produced by neutralization. 1 L of toluene was added to the resultant, and insoluble components were removed by filtration, followed by removing toluene from the filtrate using an evaporator. The resultant was distilled under reduced pressure to produce 22.0 g (0.19 mol, yield percentage: 22%) of 2,3-dihydrothiophene 1,1-dioxide (130 to 135° C./2 mmHg) represented by the following formula (6).

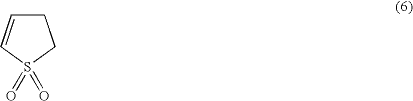

### Synthesis of 3-thiatricyclo[5.2.1.0^{2,6}]dec-8-ene 3,3-dioxide

15 g (0.13 mol) of 2,3-dihydrothiophene 1,1-dioxide, 12.6 g (0.19 mol) of cyclopentadiene, and 50 g (0.54 mol) of toluene were put into an autoclave, heated at 180° C., and reacted at a pressure of 0.3 MPa for 8 hours. After the reaction was ended, the reactant was put into a 300 ml egg-type flask, followed by removing toluene using an evaporator. The resultant was subjected to column chromatography for purification to obtain 15.2 g (0.0082 mol, yield percentage: 65%) of 3-thiatricyclo[5.2.1.0^{2,6}]dec-8-ene 3,3-dioxide represented by the following formula (7).

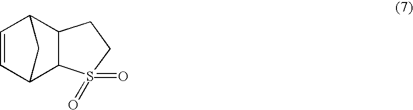

### Synthesis of 3,3-dioxide-3-thiatricyclo[5.2.1.0^{2,6}]decyl-8- and -9-formates

A 50 ml egg-type flask equipped with a reflux cooling tube and a stirrer bar was charged with 10.0 g (0.0543 mol) of 3-thiatricyclo[5.2.1.0^{2,6}]dec-8-ene 3,3-dioxide, and then a mixture of 17.5 g (0.380 mol) of formic acid and 1.33 g (0.0136 mol) of sulfuric acid was added thereto. The mixture was heated at 100° C. and reacted for 2 hours. After the reaction was ended, the reactant was cooled to room temperature and then diluted with 300 ml of toluene, followed by neutralizing with a saturated sodium hydrogen carbonate aqueous solution. Under reduced pressure, toluene and formic acid were removed from an organic layer to obtain 10.0 g (yield percentage: 80%) of a mixture of 3,3-dioxide-3-thiatricyclo[5.2.1.0^{2,6}]decyl-8- and -9-formates, represented by the following formula (8).

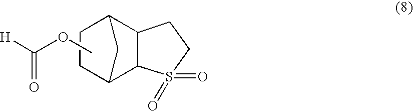

### Synthesis of mixture of 3,3-dioxide-3-thiatricyclo[5.2.1.026]decyl-8- and -9-methacrylates

A 50 ml egg-type flask equipped with a cooling tube and a stirrer bar was charged with a mixture composed of 5.0 g (0.0218 mol) of the mixture of 3,3-dioxide-3-thiatricyclo[5.2.1.0^{2,6}]decyl-8- and -9-formates, 0.74 g (0.00218 mol) of butyl titanate, and 15.22 g (0.152 mol) of methyl methacrylate. The mixture was heated at 95° C. and reacted for 2 hours. After the reaction was ended, 0.16 g (0.157 mol) of water was added, and the produced precipitate was removed by filtration. Under reduced pressure, methyl methacrylate was removed from the filtrate. The resultant was subjected to column chromatography for purification to obtain 5.86 g (0.0217 mol, yield percentage: 75%) of a mixture of 3,3-dioxide-3-thiatricyclo[5.2.1.0^{2,6}]decyl-8- and -9-methacrylates, represented by the following formula (9). The obtained product was checked by ¹H-NMR using CDCl₃ as a deuteration solvent.
(6.06-6.07 (H₂C═C<, 1H, s), 5.54-5.58 (H₂C═C<, 1H, s), 5.42-5.44, 4.92-4.94, 4.62-4.64, 4.51-4.53 (COO—CH—, 1H, d), 3.30-3.42 (>CHSO₂CH₂—, 1H, m), 2.68-3.26 (CHSO₂CH₂—, 2H, m), 1.96-1.98 (H₂C═C(CH₃)—, 3H, s), 2.20-2.40, 1.95-1.38 (>CH—, —CH₂—, 9H, m) 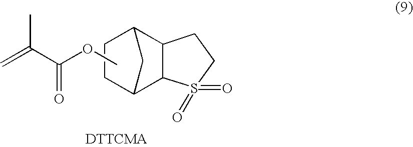
Preparation of Polymer

A glass reactor equipped with a stirring apparatus was charged with 4.0 g of the mixture of 3,3-dioxide-3-thiatricyclo[5.2.1.0^{2,6}]decyl-8- and -9-methacrylates (DTTCMA) represented by the above-mentioned formula (9), 0.0024 g of 2,2′-azobis(4-methoxy-2,4-dimethylvaleronitrile), and 12 g of dimethyl sulfoxide, and the air in the reactor was replaced by nitrogen gas. The inside of the reactor was gradually heated, and the mixture was reacted at 55° C. for 6 hours and then reacted at 70° C. for 2 hours. After the reaction was ended, the reactant was put into 1000 ml of water, and the produced precipitate was collected by filtration. The collected precipitate was dried in a vacuum at 80° C. to obtain a white solid polymer (yield point: 3.02 g; yield percentage: 75%).

A portion of the obtained polymer was dissolved in N,N-dimethylformamide, and subjected to gel permeation chromatography (GPC) using polyethylene oxide as a standard substance to determine the molecular weight thereof. The number average molecular weight Mn was 74,500, the weight-average molecular weight Mw was 124,000, and the molecular weight distribution Mw/Mn was 1.66. The physical properties of the obtained polymer are shown in Table 1.

### Example 2

A glass reactor equipped with a stirring apparatus was charged with 10 g of 1,1-dioxidetetrahydrothiene-3-ylmethacrylate (DTHTMA) represented by the following formula (10), 0.005 g of 2,2′-azobis(4-methoxy-2,4-dimethylvaleronitrile), and 30 g of dimethyl sulfoxide, and the air in the reactor was replaced by nitrogen gas. The inside of the reactor was gradually heated, and then the mixture was reacted at 55° C. for 6 hours, and then reacted at 70° C. for 2 hours. After the reaction was ended, the reactant was put into 1500 ml of water, and the produced precipitate was collected by filtration. The collected precipitate was dried in a vacuum at 80° C. to obtain a white solid polymer (yield point: 8.61 g; yield percentage: 86%). In the same way as that of Example 1, the molecular weight was determined. The number average molecular weight Mn was 173,000, the weight-average molecular weight Mw was 365,000, and the molecular weight distribution Mw/Mn was 2.11. The physical properties of the obtained polymer are shown in Table 1.

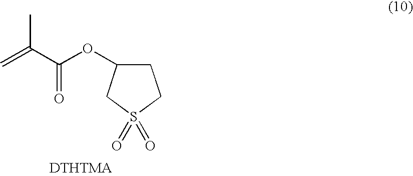

### Example 3

A three-necked flask equipped with a reflux cooling tube, a stirrer bar, and a thermometer was charged with 20 g (0.20 mol) of tetrahydropyran-4-one, 15.7 g (0.24 mol) of zinc powder, and 56.2 g of tetrahydrofuran, and the mixture was stirred under a nitrogen atmosphere at room temperature. To this solution, a mixture composed of 51.4 g (0.27 mol) of ethyl α-bromomethyl acrylate, 0.05 g (4.5×10⁻⁴ mol) of hydroquinone, and 50 g (0.78 mol) of tetrahydrofuran was added dropwise over 30 minutes. At this time, it was checked that the temperature inside the three-necked flask did not exceed 45° C.

After the end of dropwise addition, the solution was reacted for 6 hours at 45° C. by heating it in an oil bath. After the solution was cooled to room temperature, the solution was cooled to 0° C. 250 ml of a 10% hydrochloric acid aqueous solution was slowly added dropwise and then the solution was stirred for 30 minutes.

After the end of stirring, 300 ml of methylene chloride was added to the solution, and extraction was carried out. An organic layer obtained by extraction was neutralized with a saturated sodium hydrogen carbonate aqueous solution. The neutralized organic layer was concentrated using an evaporator, and the residue was distilled under reduced pressure to obtain 20.5 g (0.12 mol, yield percentage: 61%) of 3-methylene-1,8-dioxaspiro[4,5]decan-2-one (84 to 87° C./0.3 mmHg), represented by the following formula (11). The obtained product was checked by ¹H-NMR using CDCl₃ as a deuteration solvent.
(6.21-6.32 (H₂C═C—, 1H, Hₐ(3′), s), 5.63-5.74 (H₂C═C—, 1H, H_{b}(3′), s), 3.7-3.95 (—H₂COCH₂—, 4H, H(7), H(9), m), 2.80 (H₂C═C—CH₂—, 2H, H(4), s), 1.74-1.95 (—H₂C—C—CH₂—, 4H, H(6), H(10), t)

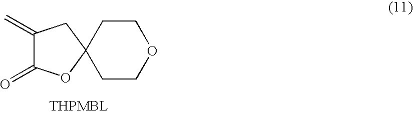

A glass reactor equipped with a stirring apparatus was charged with 2 g of 3-methylene-1,8-dioxaspiro[4,5]decan-2-one (THPMBL) prepared in the above-mentioned synthesis example and 0.008 g of 2,2′-azobis(2,4-dimethylvaleronitrile) as monomers, and the air in the reactor was replaced by a nitrogen gas. The inside of the reactor was gradually heated, and the mixture was reacted at 80° C. for 4 hours, and then reacted at 100° C. for 4 hours. After the end of the reaction, the polymer was dissolved in 30 ml of chloroform, and then put into 300 ml of n-hexane to produce a precipitate. The precipitate was collected by filtration. The collected precipitate was dried in a vacuum at 80° C. to obtain a white solid material. The yield point was 1.46 g (yield percentage: 73%). A portion of the obtained polymer was dissolved in N,N-dimethylformamide and subjected to GPC using polystyrene as a standard substance to determine the molecular weight thereof. As a result, it was revealed that the number average molecular weight Mn was 20,000, the weight-average molecular weight Mw was 39,200, and the molecular weight distribution Mw/Mn was 1.96. The polymer was soluble in chloroform or N,N-dimethylformamide, but insoluble in n-hexane, toluene, or acetone. The physical properties of the obtained polymer are shown in Table 1.

### Example 4

A white solid polymer was obtained in a similar way to that of Example 3, except that 0.5 g of THPMBL and 1.5 g of methyl methacrylate (MMA) were used as monomers. The yield thereof was 1.22 g (61%). The remaining procedures were performed in the same way as that of Example 3. The number average molecular weight Mn was 23,000, the weight-average molecular weight Mw was 45,500, and the molecular weight distribution Mw/Mn was 1.98. The polymer was soluble in chloroform or N,N-dimethylformamide, but insoluble in n-hexane, toluene, or acetone. The physical properties of the obtained polymer are shown in Table 1.

### Comparative Example 1

A glass reactor equipped with a stirring apparatus was charged with 2.0 g of methyl methacrylate (MMA) and 0.008 g of 2,2′-azobis(2,4-dimethylvaleronitrile), and the air in the reactor was replaced by a nitrogen gas. The inside of the reactor was gradually heated, and the mixture was reacted at 80° C. for 4 hours, and then reacted at 100° C. for 4 hours. After the end of the reaction, the produced polymer was dissolved in 30 ml of chloroform, and put into 300 ml of n-hexane to produce a precipitate. The precipitate was collected by filtration. The collected precipitate was dried in a vacuum at 80° C. to obtain a white solid polymer (yield point: 1.62 g; yield percentage: 81%). A portion of the obtained resin was dissolved in chloroform, and then subjected to GPC using polystyrene as a standard substance to determine the molecular weight thereof. As a result, it was revealed that the number average molecular weight Mn was 58,000, the weight-average molecular weight Mw was 154,000, and the molecular weight distribution Mw/Mn was 2.65. The physical properties of the obtained polymer are shown in Table 1.

TABLE 1

| | | | | | | | Water |
|---|---|---|---|---|---|---|---|
| | | | | | Refractive | Abbe's | absorption |
| | Relative proportions (% by mass) | | | | index | number | percentage |
| | DTTCMA | DTHTMA | THPMBL | MMA | (nD) | (νD) | (%) |
| Example 1 | 100 | 0 | 0 | 0 | 1.524 | 60 | Unmeasured |
| Example 2 | 0 | 100 | 0 | 0 | 1.514 | 61 | 2.3 |
| Example 3 | 0 | 0 | 100 | 0 | 1.532 | 58 | 2.4 |
| Example 4 | 0 | 0 | 18 | 82 | 1.502 | 57 | 0.9 |
| Comparative | 0 | 0 | 0 | 100 | 1.491 | 56 | 0.5 |
| Example 1 | | | | | | | |

In comparison with the polymer that is not according to the present invention (prepared in Comparative Example 1), the polymers according to the present invention (prepared in Examples 1, 2, 3, and 4) had high refractive index and Abbe's number, and the balance between the refractive index and Abbe's number thereof was favorable.

### Example 5

A glass reactor equipped with a stirring apparatus was charged with 2 g (0.0073 mol) of 1-adamantyl α-trifluoromethyl acrylate (AdTFMA) represented by the following formula (12), 4.67 g (0.047 mol) of methyl methacrylate (MMA), 0.0015 g of azobisisobutylonitrile, and 5 ml of toluene, and the air in the reactor was replaced by a nitrogen gas.

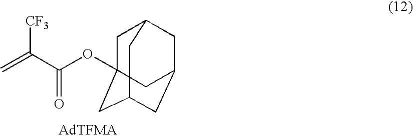

The inside of the reactor was gradually heated, and the mixture was reacted at 80° C. for 6 hours while stirring. After the end of the reaction, the reactant solution was put into 200 ml of n-hexane to produce a precipitate, and the precipitate was collected by filtration. The collected precipitate was dried in a vacuum at 80° C. to obtain a white solid polymer. The yield thereof was 4.20 g (84%).

A portion of the obtained resin was dissolved in chloroform, and then subjected to GPC using polystyrene as a standard substance to determine the molecular weight thereof. As a result, it was revealed that the number average molecular weight Mn was 63,000, the weight-average molecular weight Mw was 100,000, and the molecular weight distribution Mw/Mn was 2.04. The polymer's relative proportions (AdTFMA)/(MMA) determined by measurement of nuclear magnetic resonance (NMR) spectrum was approximately 18/82 mol %/mol %.

The refractive index was 1.502 and the Abbe's number was 84. The saturated water absorption percentage was 1.3%. The polymer was soluble in chloroform, toluene, or acetone, but insoluble in n-hexane. The physical properties of the obtained fluorine-containing copolymer are shown in Table 2.

### Comparative Example 2

The physical properties of methyl methacrylate were also evaluated in the same way as that of Example 5.

TABLE 2

| | | | | | | Saturated |
|---|---|---|---|---|---|---|
| | | | | | | water |
| | | | Refractive | Abbe's | Total light | absorption |
| | Relative proportions | | index | number | transmission | percentage |
| | AdTFMA | MMA | (nD) | (νD) | (%) | (%) |
| Example 5 | 18 | 82 | 1.502 | 84 | 86 | 1.3 |
| Comparative | 0 | 100 | 1.492 | 56 | 93 | 2.0 |
| Example 2 | | | | | | |

As is apparent from Table 2, the copolymer according to the present invention exhibited a higher refractive index and Abbe's number and lower water absorption percentage than those of methyl methacrylate, while exhibiting the same level of total light transmission as that of methyl methacrylate.

### Example 6

### Preparation of Rod Lens Array

Polymethylmethacrylate with a diffraction efficiency [η] of 0.40 (measured in MEK at 25° C.) was used. 1-Hydroxycyclohexylphenylketone was used as a photosetting catalyst and hydroquinone (HQ) was used as a polymerization inhibitor.

The physical properties were measured as follows.

<Refractive Index Distribution>

An interpha-ko interference microscope manufactured by Carl Zeiss, Inc., was used for measurement.

<Conjugation Length (Tc) and Resolution (MTF)>

Light was directed from a light source to a rod lens array through a grating pattern with a spatial frequency of 12 (line pair/mm, Lp/mm), the rod lens array having both polished end faces vertical to an optical axis. The projected grating image was detected using a charge-coupled device (CCD) line sensor provided on the imaging surface, and the maximum value (imax) and the minimum value (imin) of the measured quantity of light thereof were measured, followed by calculating modulation transfer function (MTF) in accordance with the following formula:
MTF(%)={(imax−imin)/(imax+imin)]×100.

At that time, the distance between the grating pattern and an incident end of the rod lens array was adjusted to be equal to the distance between an emitting end of the rod lens array and the CCD line sensor. Then, the grating pattern and the CCD line sensor were symmetrically moved with respect to the rod lens array to measure MTF. The distance between the grating pattern and the CCD line sensor at the time when the MTF became maximum was determined as a conjugation length.

Preparation of Original Plastic Rod Lens Yarn

Stock solutions for forming each layer were prepared as shown in the following Table 3.

TABLE 3

| | | | | | | Physical properties | |
|---|---|---|---|---|---|---|---|
| | | | | | | after curing | |
| | | | | | | Refractive | Abbe's |
| | | Components of stock solution (% by mass) | | | | index | number |
| | | PMMA | 8FM¹⁾ | DTHTMA | BzMA²⁾ | (nD) | (νD) |
| Example | First | 60.8 | 3.4 | 35 | 0.8 | 1.498 | 57.7 |
| 6 | layer | | | | | | |
| | Second | 71.6 | 6.6 | 21 | 0.8 | 1.492 | 57.2 |
| | layer | | | | | | |
| | Third | 75.6 | 12 | 11 | 1.4 | 1.487 | 56.9 |
| | layer | | | | | | |
| | Fourth | 70.5 | 21.8 | 3.5 | 4.2 | 1.479 | 56.2 |
| | layer | | | | | | |
| | Fifth | 42 | 46 | — | 12 | 1.464 | 55.0 |
| | layer | | | | | | |

¹⁾8FM = 2,2,3,3,4,4,5,5-octafluoropentylmethacrylate
²⁾BzMA = benzylmethacrylate

In order to suppress the crosstalk light or flare light, a dye Blue ACR (manufactured by NIPPON KAYAKU CO., LTD.), a dye Blue 4G (manufactured by Mitsubishi Chemical Corporation), a dye MS Yellow HD-180 (manufactured by Mitsui Toatsu Senryo Co., Ltd.), a dye MS Magenta HM-1450 (manufactured by Mitsui Toatsu Senryo Co., Ltd.), and a dye KAYASORB CY-10 (manufactured by NIPPON KAYAKU CO., LTD.) were added to the stock solution for the fifth layer before being heated and kneaded, the content of each with respect to the total mass of the stock solution being shown in the following table.

TABLE 4

| Dye | Fifth layer (% by mass) |
|---|---|
| Blue ACR | 0.571 |
| Blue 4G | 0.011 |
| MS Magenta HM-1450 | 0.143 |
| MS Yellow HD-180 | 0.143 |
| KAYASORB CY-10 | 0.011 |

Then, the stock solutions for each layer were heated at 70° C. and kneaded, followed by being arrayed so that each layer's cured form had a gradual decrease of the refractive index from the central portion, and then being simultaneously extruded from a concentric five-layer conjugate spinning nozzle. The temperature of the conjugate spinning nozzle was 50° C. The ejection ratio of the stock solutions for each layer, the ratio being converted into the thickness ratio of each layer in a radial direction of the plastic rod lens (that is, the thickness of the first layer is radius), the first layer/the second layer/the third layer/the fourth layer/the fifth layer, was 6/40/33/17/4.

Then, a filament extruded from the conjugate spinning nozzle was drawn up with a nip roller (200 cm/minute) to pass through a mutual diffusion processing part with a length of 30 cm, and then pass centrally in a first curing part (first light irradiation part) with a length of 60 cm in which 18 chemical lamps with a power of 20 watt were provided in upper and lower stages of the circumference of the continuous center axis with constant intervals to be cured, and then further pass centrally in a second curing part (second light irradiation part) in which three high-pressure mercury lamps with a power of 2.0 kilowatt were provided in the circumference of the center axis with constant intervals to be completely cured. The flow rate of a nitrogen gas in the mutual diffusion processing part was 80 L/minutes. The radius of the produced original plastic rod lens yarn was 0.295 mm.

An original plastic rod lens yarn prepared in the same way except that the outer circumference portion does not include any dyes had a central refractive index of 1.493 and an outer circumference refractive index of 1.471, and thus the refractive index thereof continuously decreased toward the outer circumference. If dyes are included, the refractive index distribution is assumed to be the same.

Preparation of Plastic Rod Lens

The original plastic rod lens yarn was drawn by 2.71 times under an atmosphere of 135° C., and subjected to relaxing processing under an atmosphere of 115° C. so that the relaxation rate was 434/542.

The radius of the produced plastic rod lens was 0.2 mm, and the central refractive index thereof was 1.493. The refractive index distribution thereof approximated to the formula (1) in the region where the distance from the center axis to the outer circumference direction was 0.2r to 0.8r, and the distribution constant g of the refractive index was 0.84 mm⁻¹ at a wavelength of 525 nm.

A layer with a thickness of approximately 20 μm, in which dyes were approximately uniformly present, was formed from the outer circumference surface to the central direction.

Preparation of Rod Lens Array

Plural plastic rod lenses produced were closely and parallel arrayed in a row (with intervals of 0.4 mm) between two base plates made from a phenol resin, and spaces therebetween were filled with an adhesive (ARALDITE RAPID), followed by curing the adhesive between the plastic rod lenses and between the plastic rod lens and the base plate. Then, both end surfaces vertical to center axes of the plastic rod lenses were mirror-finished using a diamond blade to produce a rod lens array in which the length of the plastic rod lens was 4.4 mm. The conjugation length Tc of the rod lens array measured at a wavelength of 525 nm was 10.0 mm and the MTF thereof at that time was 50%.

Each value of Tc of the rod lens array was determined by performing measurement at a wavelength of 470 nm, 525 nm, and 630 nm (Table 5). As a result of comparison of each of the minimum values of MTF measured at the three wavelengths by setting the length between the grating pattern and the CCD line sensor to each Tc determined above, it was revealed that when a value of Tc was 10.1 mm, the minimum value of MTF measured by setting the length to the value of Tc was maximum. Each value of MTF measured at each of the wavelengths by setting the length to the value of Tc, the value being 10.1 mm and realizing the most excellent color characteristics, was measured (Table 6).

TABLE 5

Tc (mm)

| | 470 nm | 525 nm | 630 nm | ΔTc |
|---|---|---|---|---|
| Example 6 | 9.9 | 10.0 | 10.1 | 0.2 |

TABLE 6

MTF (%)

| | 470 nm | 525 nm | 630 nm |
|---|---|---|---|
| Example 6 | 42 | 48 | 42 |

The chromatic aberration ΔTc (difference between Tc (470) and Tc (630)) of the rod lens array prepared in Example 6 was 0.2 mm, which was small.

### INDUSTRIAL APPLICABILITY

The polymer according to the present invention has a refractive index and Abbe's number higher than those of polymethylmethacrylate, and the balance between the refractive index and Abbe's number is favorable. Accordingly, the polymer is excellent as a raw resin used for an optical component such as a plastic lens, plastic optical fiber, plastic rod lens, optical waveguide, or the like, or an optical resin such as a resin used for a disc, resin used for a light-emitting diode, a resin used for a transparent electrode, a resin used for a liquid crystal display, or the like. The compound according to the present invention can be used as a raw material for such a polymer.

Also, the rod lens array in which the rod lenses containing such a polymer are fixedly arrayed has a high resolution, and is particularly suitable for a copying machine, facsimile, printer or scanner.

1. A compound represented by a formula (1-a): 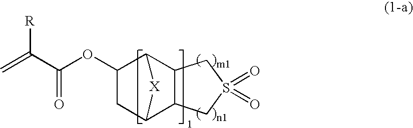
wherein R represents a hydrogen atom or CH₃, X represents —CH₂—, —O—, or —SO₂—, l represents 1 or 2, and m1 and n1 each represents an integer of 0 or more, provided that a sum of m1 and n1 is 2.

2. A polymer comprising a structural unit based on a compound (A-a) represented by a formula (1-a): 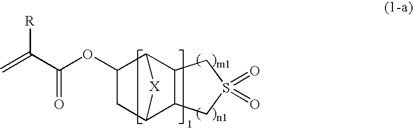
wherein R represents a hydrogen atom or CH₃, X represents —CH₂—, —O—, or —SO₂—, l represents an integer of 1 or 2, and m1 and n1 each represents an integer of 0 or more, provided that a sum of m1 and n1 is 2, or a formula (3): 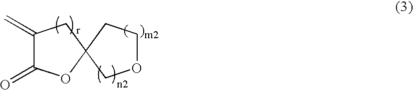
wherein r represents 1 or 2, m2 represents an integer of 0 to 2, and n2 represents an integer of 1 to 3, provided that a sum of m2 and n2 is 2 or 3, a hydrogen atom in —CH₂— being optionally substituted by another functional group.

3. A polymer according to claim 2, further comprising a structural unit based on a vinyl monomer (B).

4. A polymer according to claim 2, wherein a refractive index is 1.500 to 1.600, and an Abbe's number is 56 to 70.

5. An optical component comprising the polymer of claim 2.

6. A plastic rod lens comprising the polymer of claim 2.

7. A rod lens array comprising: two base plates; and a plurality of the plastic rod lens of claim 6, fixedly arranged in such a way that each plastic rod lens has a center axis directed almost in parallel with each other between the two base plates.

8. An optical component comprising a polymer comprising a structural unit based on a compound (A-b) represented by a formula (1-b): 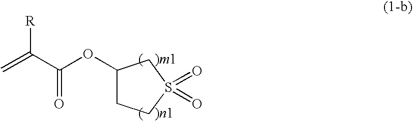
wherein R represents a hydrogen atom or CH₃, X represents —CH₂—, —O—, or —SO₂—, and m1 and n1 each represents an integer of 0 or more, provided that a sum of m1 and n1 is 2.

9. An optical component comprising a polymer comprising a structural unit based on a compound (A-b) represented by a formula (1-b) and a structural unit based on a vinyl monomer (B): 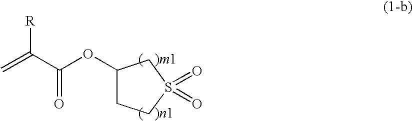
wherein R represents a hydrogen atom or CH₃, X represents —CH₂—, —O—, or —SO₂—, and m1 and n1 each represents an integer of 0 or more, provided that a sum of m1 and n1 is 2.

10. A plastic rod lens comprising a polymer comprising a structural unit based on a compound (A-b) represented by a formula (1-b): 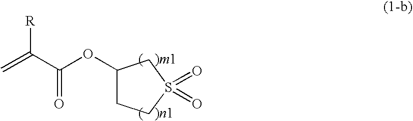
wherein R represents a hydrogen atom or CH₃, X represents —CH₂—, —O—, or —SO₂—, and m1 and n1 each represents an integer of 0 or more, provided that a sum of m1 and n1 is 2.

11. A plastic rod lens comprising a structural unit based on a compound (A-b) represented by a formula (1-b) and a structural unit based on a vinyl monomer (B): 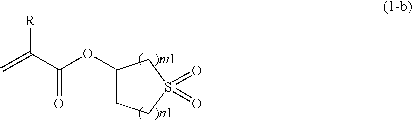
wherein R represents a hydrogen atom or CH₃, X represents —CH₂—, —O—, or —SO₂—, and m1 and n1 each represents an integer of 0 or more, provided that a sum of m1 and n1 is 2.

12. A rod lens array comprising: two base plates; and a plurality of the plastic rod lens of claim 10, fixedly arranged in such a way that each plastic rod lens has a center axis directed almost in parallel with each other between the two base plates.

13. An optical component comprising a polymer having a refractive index within a range of 1.500 to 1.600 and an Abbe's number within a range of 56 to 70.

14. A plastic rod lens comprising a polymer having a refractive index within a range of 1.500 to 1.600 and an Abbe's number within a range of 56 to 70.

15. A rod lens array comprising: two base plates; and a plurality of the plastic rod lens of claim 14, fixedly arranged in such a way that each plastic rod lens has a center axis directed almost in parallel with each other between the two base plates.
